(19) United States
(12) Patent Application Publication
(10) Pub. No.: US 2023/0225389 A1
(43) Pub. Date: Jul. 20, 2023

(54) PROLONGED RELEASE FORMULATION OF CAFFEINE

(71) Applicant: INVENTIA HEALTHCARE LIMITED, Andheri (East), Mumbai (IN)

(72) Inventors: Vishal SHAH, Vile Parle (West), Mumbai (IN); Rajat SHAH, Vile Parle (West), Mumbai (IN); Shajahan ABDUL, Madurai (IN)

(21) Appl. No.: 18/002,440

(22) PCT Filed: Dec. 11, 2020
(86) PCT No.: PCT/IB2020/061795

§ 371 Date: Dec. 19, 2022

(30) Foreign Application Priority Data
Jul. 31, 2020 (IN) 202021032839

(51) Int. Cl. A23L 33/00; A23L 29/00; A23L 2/38; A23L 2/395

(52) U.S. Cl. CPC A23L 33/30; A23L 29/045; A23L 2/38; A23L 2/395

(57) ABSTRACT

The present invention relates to a prolonged release formulation of caffeine, which comprises of caffeine embedded in a continuous phase of non-polymeric release controlling carrier. The formulation may be optionally comprised of one or more excipients which are acceptable in nutraceutical and food industry. The invention also relates to a process for preparation; wherein caffeine is uniformly distributed throughout the continuous phase of non-polymeric release controlling carrier to get single-phase granules. The formulation releases caffeine through continuous phase of non-polymeric release controlling carrier, wherein about 20 to 60% of caffeine is released in first hour, followed by about 50-90% of release in 4-8 hours and about 70 to 100% of caffeine release in 8-12 hours. Caffeine formulation can advantageously be administered to a subject for achieving stimulant effect through prolonged release of caffeine over entire day.

The present invention relates to a prolonged release formulation of caffeine, which comprises of caffeine embedded in continuous phase of non-polymeric release controlling carrier. The continuous phase may be comprised of about 20 to 70% by weight of caffeine and about 20 to 80% by weight of non-polymeric release controlling carrier. The formulation may be optionally comprised of one or more excipients, which are acceptable in nutraceutical and food industry. The invention also relates to a process for preparation; wherein caffeine is uniformly distributed throughout the continuous phase of non-polymeric release controlling carrier to get single-phase granules. The formulation releases caffeine through continuous phase of non-polymeric release controlling carrier, wherein about 25 to 60% of caffeine is released in first hour, about 50 to 90% of release in 4-8 hours and about 70 to 100% of caffeine release in 8-12 hours. Caffeine formulation comprising single-phase granules can advantageously be administered to a subject for achieving stimulant effect, improving the focus and conferring the alertness through prolonged release of caffeine over entire day.

### BACKGROUND OF THE INVENTION

Caffeine is the most frequently consumed stimulant in the world which belongs to the class of xanthine or xanthine derivative. Approximately 80 percent of the adult population consumes between 200-250 mg of caffeine daily. Conveniently, caffeine is found in beverages such as coffee, tea and soda as well as certain foods such as chocolate and cocoa, so that an individual merely has to consume such foods or beverages in order to introduce caffeine into their system. One of the most common forms of administering caffeine is by consuming a cup of hot coffee early in the morning to help the individual to become more alert when beginning the day. Many consume three to four cups of coffee a day, to maintain caffeine levels in the body throughout the day. Moderate doses of caffeine can lead to an increase in physical task performance, making it very useful for combating fatigue. Caffeine is also believed to have other beneficial uses as well including improving body coordination, endurance, and mental performance.

Caffeine's effect is likely attributable to its function as a mild central nervous system stimulant, whereby it competitively binds to adenosine receptors, leading to a suppression of its inhibitory effect on CNS activity. Caffeine is used in many commercially available energy supplements, which is consumed by sports person as well as students. Such products typically induce only 90-120 minutes of increased alertness and consequently, repeat administration of such energy supplements is practiced. This is because caffeine is only active for a short period of time and then its half-life reduces its effectiveness.

Moreover, the frequent use of such products can have adverse effects on resting cardiovascular parameters, as well as on hepatic and renal function. Higher and frequent doses also cause other uncomfortable side effects such as anxiety, jitters, and result in the difficulty of relaxing and sleeping.

Prior art deals with developing sustained or slow release formulations of caffeine, in order to modulate release of caffeine to achieve its effect in desired hours of the day. Many of the formulations are designed in the form of combination of immediate release and extended release, to achieve initial loading dose for providing alertness, followed by extended release of the active over desired time period. The formulations are mostly in the form of core coated with hydrophilic or hydrophobic polymer; or in the form of microparticles in which active is layered on neutral core, followed by multiple layers of coatings to control release of the active. Such type of formulations requires specialized equipment and the migration and release of active becomes dependent on multiple factors such as core, coating layers, thus resulting into complexity in achieving in-vitro and in-vivo relationship for release of active. More precisely, there are chances of dose dumping or incomplete drug release from such dosage forms, thus ultimately affecting the efficacy of the formulation and failure in getting desired effect.

U.S. Pat. No. 8,268,352 relates to modified release dosage form comprising of micromatrix particles of a high solubility active ingredient prepared by using dual retard technique, which is a combination of matrix formulation and reservoir formulation. The dosage form consists of micro matrix particles which contain active ingredient and one or more hydrophobic release controlling agents. The particles are further coated by hydrophobic release controlling agents to form a double barrier to control the diffusion of the active.

U.S. Pat. No. 5,700,484 describes a sustained release composition in the form of microparticle comprising of three layers surrounding the core. The first layer surrounding the core comprises of a biodegradable matrix system comprising a water insoluble release retardant, stimulant and a water-soluble binding agent which binds the stimulant to the microparticle; the second layer surrounds the first layer and further delays the release; third layer is similar to the first layer comprising of water-soluble binding agent, a water insoluble release retarding agent and stimulant. The water-soluble polymers used herein are polyvinyl pyrrolidones or similar water-soluble organic polymers such as polyacrylate esters.

US20160128943 relates to an oral formulation comprising a first component providing for immediate or rapid release of caffeine, and a second component providing for extended release of caffeine, wherein the formulation provides a blood plasma concentration of caffeine at least about 0.3 μg/mL and maintains the concentration for at least four hours. The extended release component of the formulation comprises one or more hydrophilic polymers.

US20190192444 describes a composition comprising at least one microparticle, wherein said at least one microparticle is in the form of a core-shell, and wherein said core comprises at least one active agent; and at least one pH-triggered ingredient, at least one enteric ingredient, or at least one enzymatically-degradable ingredient, or any combination thereof; and said shell comprises one or more water-insoluble and enzymatically-degradable ingredients, wherein the weight ratio of said active agent and the sum of (b) ranges from 1:10 to 10:1.

U.S. Pat. No. 8,449,920 relates to a method for producing sports beverage comprising: providing one or more sustained-release beads, each of the sustained-release beads comprising, 40 wt % to 95 wt % of one or more binding agents; and 5 wt % to 60 wt % of active ingredients consisting of caffeine, electrolytes, and vitamins; providing a dispersion medium comprising: an aqueous solution; and one or more thickening agents; and mixing the one or more sustained-release beads in the dispersion medium.

There is a need to develop caffeine formulations, which would ensure reliable, reproducible, and consistent release pattern of the active for prolonged time of the day, with low variability. Such formulations should help the subject to achieve the desired level of alertness during initial hours in the day and prolonging the effect throughout the day, without interfering with normal sleep cycle.

It was surprisingly found, that these requirements were met by formulations of the present invention, by employing non-coated, non-layered, single-phase granule composition, comprised of non-polymeric release controlling carrier in which caffeine is uniformly dispersed, using simple and economic process of melt granulation. These formulations are easy to prepare, using commonly available equipment. Optimal selection of type and concentration of non-polymeric release controlling carrier by the present inventors has resulted in continuous phase formulation of caffeine which ensures slow release of active over about 6-12 hours. It would thus be advantageous to provide caffeine in a prolonged release continuous phase formulation that does not require re-administration of the active, for maintaining the alertness throughout the day.

### SUMMARY OF THE INVENTION

The present inventors have developed a prolonged release formulation of caffeine for oral administration, which is non-coated and non-layered. The formulation is comprised of caffeine embedded in continuous phase of at least one non-polymeric release controlling carrier. The composition may be comprised of about 20 to 70% by weight of caffeine and about 20 to 80% by weight of continuous phase comprised of at least one non-polymeric release controlling carrier and optionally about 0 to 60% by weight of one or more excipients, which are acceptable in nutraceutical and food industry.

The invention also provides process for preparation of caffeine formulation; wherein caffeine is uniformly distributed throughout the continuous phase of non-polymeric release controlling carrier by method of melt granulation. The formulation of the invention is non-coated, non-layered, single-phase formulation of caffeine.

The granules of caffeine may range in size from 100 micron to 1500 micron; preferably 150 to 1000 micron and more preferably from 400 to 1000 micron. The granules prepared in this way are free flowing and can be compressed in tablets, filled in capsules, can be added in beverages, formulated as chewing gum and gummies or used as such in sachets and stick packs for convenient oral administration to the subjects.

The formulation releases caffeine over a prolonged period of about 8-12 hours, wherein about 25 to 60% of caffeine is released in first hour, followed by about 50-90% of release in 4-8 hours and about 70 to 100% of caffeine release in 8-12 hours.

The prolonged release formulation of caffeine provides slow release of caffeine over a longer period, to impart an increased level of alertness, over 6-12 hours, likely eliminating the need for repeat administration. The process for preparation is easy, economic and also makes use of commonly available industrial equipment.

### Objectives of the Invention

The main objective of the invention is to provide a prolonged release formulation, in which caffeine is uniformly embedded in continuous phase of non-polymeric release controlling carrier.

Another objective is to provide a non-coated, non-layered, single-phase formulation which is comprised of about 20 to 70% by weight of caffeine and about 20 to 80% by weight of non-polymeric release controlling carrier. The formulation may optionally be comprised of about 0-60% by weight of one or more excipients, which is acceptable in nutraceutical and food industry.

One important objective is to provide caffeine prolonged release formulation which is comprised of continuous phase of non-polymeric release controlling carrier obtained from natural source, selected from the group of, but not limited to fatty acids, long chain alcohols, fats, lipids, waxes, oils and the combination thereof. Continuous phase may also optionally be comprised of excipients selected from the group of, but not limited to diluent, filler, binder, disintegrant, anti-adherent, glidant, gum base, lubricant, pH adjusting agent, channelizing agent, sweetening agent and the combination thereof.

One important objective of the present invention is to provide caffeine formulation which releases caffeine over a period of 8-12 hours, wherein about 25 to 60% of caffeine is released in first hour, about 50-90% of release in 4-8 hours and about 70 to 100% of caffeine release in 8-12 hours.

Another objective of the invention is to provide a process for preparation of prolonged release caffeine formulation wherein caffeine is uniformly distributed throughout the continuous phase of non-polymeric carrier, using suitable process of granulation. The granules are uniform and free flowing, which may be used as such through sachets, can be added in beverages, filled in capsules or compressed in tablets for oral administration to the subject.

One more objective of the present invention is to provide a formulation which releases caffeine from continuous phase of non-polymeric release controlling carrier, over a prolonged period of 10-12 hours, to impart alertness to the individuals throughout the day, likely eliminating the need for repeat administration.

### DETAILED DESCRIPTION

The invention relates to a prolonged release formulation of caffeine, wherein the active is uniformly distributed in continuous phase of non-polymeric release controlling carrier. Caffeine formulation is non-coated, non-layered and single-phase composition, comprised of at least one non-polymeric release controlling carrier.

The formulation may be free of any excipients or may be optionally comprised of one or more excipients, which are acceptable in nutraceutical and food industry. The invention also relates to a process for preparation, wherein caffeine is embedded uniformly in the continuous phase of non-polymeric carrier, by suitable process of granulation. Formulation releases caffeine over a prolonged period of 8 to 12 hours and it is useful for delivering the stimulant over day-long time, thus eliminating the need for repeated administration of the active.

Caffeine as used in this invention is purchased in the form of white crystalline, odorless, bitter powder and having 98 to 101% of active content. Caffeine exhibits good water solubility. It is supplied from a manufacturer, who obtains it from natural source Coffea Robusta, belonging to Rubiaceae family.

The terminology ‘prolonged release’ as used herein refers to release pattern of the active from continuous phase of non-polymeric release controlling polymer, at desired rate over a period of 6 to 12 hours. The formulation, as described herein, is designed in such a way that about 25 to 60% of caffeine is released in first hour, followed by about 50-90% of release in 4-8 hours and about 70 to 100% of caffeine release in 8-12 hours. Thus the active would be released in the body system for exerting its stimulant action through release of about 25 to 60% of caffeine in initial phase and providing alertness to the subject, followed by about 70 to 100% of release in 8-12 hours, wherein the active would be available in the body system for the entire day as per requirement.

The terminology ‘continuous phase’ as used herein refers to uniform distribution of active in continuous phase of non-polymeric release controlling carrier. In present invention, the continuous phase may be the mixture of active with non-polymeric release controlling carrier and optionally the excipients, which are acceptable in nutraceutical and food industry. The prolonged release formulation of caffeine, as described herein is non-coated, non-layered and single-phase matrix granules, preferably comprised of one or more non-polymeric release controlling carrier. Caffeine is uniformly dispersed and embedded in the continuous phase of non-polymeric release controlling carrier and optionally the excipients, to form a system through which caffeine releases at a prolonged rate over 6 to 12 hour, when the formulation enters the body system after administration.

The terminology ‘non-polymeric release controlling carrier’ as used herein relates to the formulation components which are used as continuous phase, for uniformly distributing caffeine for achieving desired prolonged release profile. These components are explicitly responsible for controlling release of caffeine from the formulation. These carriers are insoluble in water. The non-polymeric release controlling carrier may be preferably obtained from natural source, although the carriers may be available from synthetic and semi-synthetic sources.

As per one important embodiment, the non-polymeric release controlling carrier may be selected from, but not limited to, saturated fatty acids having 12 to 28 carbons, such as stearic acid, fatty alcohols having from 16 to 44 carbons, cetyl alcohol, pegylated fatty acids, glycerol fatty acid esters, monoglycerides, diglycerides, triglycerides, derivatives of mono-diglycerides, glyceryl behenate, glyceryl monostearate, glyceryl palmitostearate, pegylated vegetable oils, partially hydrogenated oils of soy, cottonseed, palm, sunflower, castor oil, sorbitan esters, polyoxyethylene sorbitan esters, propylene glycol mono- or diesters, phospholipids, phosphatides, cerebrosides, gangliosides, cephalins, lipids, glycolipids, sulfatides, sugar esters, sugar ethers, sucrose esters, sterols, polyglycerol esters, glycerolipid, phosphatic acid, phosphatidylethanolamine, phosphatidylcholine, phosphatidylserine, phosphatidylinositol or other glycerophospholipids, ceramide, sphingolipid, sterol, fat-soluble vitamin, prenol, saccharolipid, polyketide, their salts and esters and the combination thereof.

Non-polymeric release controlling carrier may be selected from the class of, but not limited to lipids, fats, waxes and the combination thereof. Non-polymeric release controlling carrier may also be selected from, but not limited to, beeswax, candelilla wax, carnauba wax, spermaceti, paraffin wax, synthetic waxes and the combination thereof.

The formulation, as described herein may comprise of about 20 to 70% by weight of caffeine and 20 to 80% by weight of non-polymeric release controlling carrier. The formulation may optionally contain about 0 to 60% by weight of excipients.

According to one more embodiment of the invention, prolonged release formulation of caffeine may be comprised of about 20 to 70% by weight of caffeine and about 22 to 80% by weight of non-polymeric release controlling carrier and optionally 0 to 50% by weight of excipients.

According to one embodiment of the invention, the formulation may be comprised of non-polymeric release controlling carrier selected from wax, saturated fatty acid, lipids, oils and the combination thereof.

As per one embodiment of the invention, continuous phase prolonged release formulation of caffeine may optionally contain 0 to 60% by weight of excipients, which are acceptable in nutraceutical and food industry. Excipients may facilitate the granulation and formulation of caffeine prolonged release formulation, but these do not have any role in controlling release of caffeine from the formulation. These are commonly used ingredient in industry, and are selected from the group of, but not limited to, fillers, diluents, disintegrants, lubricants, binders, glidants, anti-caking agents, stabilizers, surfactants, channelizing agents, vehicles, buffers, stabilizers, preservatives, acidifiers, alkalizers, complexing agents, gum bases, antioxidants, viscosity enhancers, plasticizers, coating materials, sweeteners, colors, flavors and the combination thereof. The excipients may belong to the class such as polymeric, non-polymeric, swelling, non-swelling, pH dependent and pH independent additives, which do not have any role in controlling release of active from the formulation. Prolonged release formulation of caffeine, as described herein, may be comprised of about 0 to 60% by weight of additives, which are selected from natural, semi-synthetic or synthetic sources. More preferably, the formulation may be comprised of about 0 to 50% by weight of excipients. Most preferably, the prolonged release formulation may be comprised of about 0 to 30% by weight of excipients.

The formulation may be comprised of diluents known in the art, but not limited to microcrystalline cellulose, silicified microcrystalline, cellulose, powdered cellulose, microfine cellulose, corn starch, rice bran extract, calcium phosphate, dibasic calcium phosphate, tribasic calcium phosphate, calcium sulfate, or mixtures thereof. The diluents may also be selected from glucose, lactose, sucrose, dextrose, fructose, compressible sugar, or mixtures thereof.

The binders may be selected from the group of cellulose derivatives such as hydroxypropylmethyl cellulose (HPMC), hydroxypropyl cellulose (HPC), ethylcellulose, carboxymethylcellulose (CMC), sodium CMC, potassium CMC, calcium CMC, methylcellulose, hydroxyethyl cellulose (HEC), microcrystalline cellulose; polyvinylpyrrolidone (PVP), vinyl pyrrolidone-vinyl acetate copolymer, polyvinyl alcohol, starch, carbomer, gums like xanthan gum, guar gum, acacia, locust bean gum, alginates, or mixtures thereof.

The disintegrants may be selected from sodium starch glycolate, crospovidone, croscarmellose sodium, croscarmellose calcium, croscarmellose potassium, sodium carbonate, sodium hydrogen carbonate, calcium carbonate, starch, starch 1500, modified starch, pregelatinized starch, crosslinked carboxymethyl starch, sodium hydrogen carbonate, hydroxypropyl cellulose, sodium carboxymethylcellulose or mixtures thereof.

The lubricants may be selected from magnesium stearate, calcium stearate, sodium benzoate, talc, or mixtures thereof.

The glidants may be selected from suitable glidants known in the art and commonly used in the industry, selected from the group of stearate, starch, talc and the derivatives.

According to the important embodiment of the invention, the process for preparation of caffeine formulation may be comprised of mixing caffeine with at least one non-polymeric release controlling carrier and optionally one or more excipients. The blend is subjected to suitable process of granulation such as slugging, dry granulation, direct compression, extrusion spheronization, compaction, compression, melt granulation, melt extrusion, melt solidification, spray-drying, freeze-drying and spray chilling. The formulation of caffeine, as described herein is non-coated, non-layered and continuous phase composition, which provides reliable, reproducible and consistent release of caffeine over prolonged time of 6 to 12 hours.

As per one more embodiment of the invention, commonly available and easy to use industrial equipment may be used for preparation of prolonged release formulation of caffeine. Suitable parameters of temperature, revolutions and torque may be selected for carrying out melt continuous granulation. The process may be carried out by varying the temperature in the range of 50 to 100° C. and the molten form can be processed to get granules of desired size.

According to one embodiment of the invention, size of the granules/powder may vary from 100 micron to 1500 micron; preferably from 150 micron to 1000 micron;

As per one more embodiment, the granules may vary in size from 400 micron to 800 micron. The granules prepared in this way are free flowing in nature.

Caffeine granules can be used as such, mixed in beverages, filled in the capsules, compressed in the tablets to get final dosage form for oral administration. The granules can also be formulated in chewing gums and gummies. The formulations of the present invention may be granules, tablets, capsules, caplets, pills, beads, beadlets, pellets, and the like.

The formulations comprising the said caffeine granules embedded in continuous phase of non-polymeric release controlling carrier, may optionally contain one or more excipients suitable for the preparation of the dosage form. These formulations can be manufactured by conventional processes known to a person skilled in the art.

Prolonged release caffeine formulations can be evaluated for assay, stability as well as cumulative release profile of caffeine over 12-hour time period.

According to one embodiment of this invention, caffeine formulation releases active over a prolonged period, wherein about 25 to 60% of caffeine is released in first hour, about 50-90% of caffeine release in 4-8 hours and about 70 to 100% of release in 8-12 hours.

According to one more embodiment of this invention, caffeine formulation preferably releases about 30 to 60% of caffeine in first hour, followed by about 60 to 80% of caffeine release in 4-8 hours and about 75 to 100% of release in 8-12 hours.

As per important embodiment of the invention, formulation exhibits immediate release of about 20 to 60% of caffeine in first hour, followed by about 65 to 80% of prolonged drug release in 4-8 hours and 75 to 100% release in 8-12 hours, thus providing the prolonged therapeutic effect of the active for 6 to 12 hours, as desired for achieving stimulant action of caffeine over entire day.

The prolonged release caffeine formulation of the invention can be conveniently administered to the subject for achieving stimulant action and alertness during the start of the day by initial release of active in first hour, followed by prolonged levels of caffeine throughout the day over a period of 6 to 12 hours. Thus, the formulation does not interfere with sleeping hours of the subject. These prolonged release formulations decrease the frequency of administration of the dosage form during the day.

The invention is now illustrated with non-limiting examples.

### Example 1: Composition of Caffeine Formulation-Formula 1

Caffeine (50%) was mixed with stearic acid (20%), carnauba wax (20%) and maltodextrin (10%) in a blender for 30 minutes at 10 rpm. The mixture was fed in hot melt granulator, while maintaining conditions like feed rate, temperature, and torque. The temperature was varied from 50 to 100° C. The molten form was collected and processed to get granules of Formula 1 in the range of 400 to 1000 micron.

The formulation was evaluated for dissolution profile using USP Type II (paddle) apparatus at 100 rpm speed, using 900 ml of pH 6.8 phosphate buffer as dissolution medium. Caffeine released from the formulation is measured by using UV spectrophotometer at 274 nm wavelength and the data is provided in Table no. 1.

TABLE 1
Dissolution profile of Caffeine prolonged
release formulation-Formula1

| Time (hr.) | Cumulative Release profile of Caffeine (%) |
|---|---|
| 1 | 42 |
| 2 | 57 |
| 4 | 74 |
| 6 | 83 |
| 8 | 89 |
| 10 | 93 |
| 12 | 96 |

The dissolution profile of caffeine formulation exhibits prolonged release pattern, wherein about 42% of caffeine is released in first hour and about 70 to 90% of caffeine is released in 4-8 hours, followed by about 100% release in 10-12 hours. The release pattern is achieved from single-phase granules, prepared from use of non-polymeric release controlling carriers. Caffeine formulation with continuous phase of non-polymeric carrier is useful for achieving alertness during the start of the day by initial fast release of active in first hour, followed by prolonged levels of caffeine throughout the day over a period of 10 to 12 hours. Thus the formulation can result in improved performance of the subject, through enhanced focus and alertness throughout the day.

### Example 2: Caffeine Formulation with Non-Polymeric Carrier in Various Ratios

TABLE 2
Composition of Caffeine formulations -Formula 2 to Formula 7

| | Formula 2 | Formula 3 | Formula 4 | Formula 5 | Formula 6 | Formula 7 |
|---|---|---|---|---|---|---|
| Ingredients | Percentage | Percentage | Percentage | Percentage | Percentage | Percentage |
| Caffeine | 50 | 50 | 50 | 50 | 60 | 70 |
| Stearic acid | 15 | 15 | 14 | 12 | 15 | 15 |
| Carnauba Wax | 15 | 15 | 14 | 12 | 15 | 15 |
| Maltodextrin | 20 | — | 12 | 16 | — | — |
| Microcrystalline | — | 20 | 10 | 10 | 10 | — |
| cellulose (MCC) | | | | | | |
| Total | 100% | 100% | 100% | 100% | 100% | 100% |
| Particle Size | 400 to 800 | 400 to 800 | 400 to 800 | 400 to 800 | 150 to 250 | 150 to 250 |
| | micron | micron | micron | micron | micron | micron |
| Time (hr.) | Cumulative Release Profile of Caffeine (%) over 12 hours | | | | | |
| 1 | 40 | 27 | 35 | 54 | 58 | 48 |
| 2 | 45 | 42 | 47 | 70 | 67 | 55 |
| 4 | 50 | 55 | 61 | 86 | 81 | 63 |
| 6 | 60 | 72 | 74 | 93 | 88 | 70 |
| 8 | 67 | 79 | 79 | 96 | 90 | 74 |
| 10 | 72 | 85 | 85 | 99 | 93 | 77 |
| 12 | 74 | 89 | 87 | 100 | 95 | 80 |

Process of Formula 1, as described in Example 1 was followed to prepare various caffeine formulations, covered in Table 2. The molten form was collected and processed to get granules in the range of 150 to 1000 micron. The release profile was checked using dissolution apparatus, which is also included in Table 2. The dissolution profile of caffeine formulation indicates prolonged release pattern, wherein about 25-60% of caffeine is released within first one hour and about 50 to 90% of caffeine is released in 4-6 hours, followed by about 75-100% release in 10-12 hours. The release pattern is achieved through use of continuous phase of non-polymeric release controlling carriers.

### Example 3: Caffeine Formulations with Non-Polymeric Carrier in Various Ratios

TABLE 3
Composition of Caffeine formulations -Formula 8 to Formula 11

| | Formula | | | |
|---|---|---|---|---|
| | Formula 8 | Formula 9 | Formula 10 | Formula 11 |
| | Composition in Percentage | | | |
| Caffeine | 50 | 50 | 50 | 58.9 |
| Stearic acid | 15 | 20 | 20 | 16.1 |
| Carnauba Wax | 15 | 20 | 20 | 16.1 |
| Maltodextrin | 10 | — | 10 | — |
| MCC | 10 | 10 | — | — |
| Shellac | — | — | — | 8.9 |
| Total | 100% | 100% | 100% | 100% |
| Particle size | 400-800 micron | 400-800 micron | 400-800 micron | 400-800 micron |
| Time (hr.) | Cumulative Release Profile of Caffeine (%) over 12 hours | | | |
| 1 | 54 | 51 | 42 | 44 |
| 2 | 70 | 66 | 57 | 57 |
| 4 | 86 | 80 | 74 | 71 |
| 6 | 93 | 87 | 83 | 80 |
| 8 | 99 | 90 | 89 | 85 |
| 10 | 100 | 93 | 93 | 90 |
| 12 | 102 | 95 | 96 | 93 |

Process of Formula 1, as described in Example 1 was followed to prepare various caffeine formulations, covered in Table 3. The molten form was collected and processed to get free flowing granules in the range of 400 to 800 micron. The release profile was checked using dissolution apparatus, which is also included in Table 3.

The dissolution profile of caffeine formulation indicates prolonged release pattern, wherein about 40-60% of caffeine is released within first hour and about 70 to 90% of caffeine is released in 4-6 hours, followed by about 85-100% release in 8-12 hours. The release pattern is achieved through use of continuous phase of non-polymeric release controlling carriers.

### Example 4: Caffeine Formulations with Glyceryl Behenate as Non-Polymeric Carrier

TABLE 4
Composition of Caffeine formulations -Formula 12 and 13

Formula

| | |
|---|---|
| Formula 12 | Formula 13 |

Percent Composition

| | | |
|---|---|---|
| Caffeine | 20 | 40 |
| Glyceryl Behenate | 80 | 60 |
| Total | 100% | 100% |
| Particle Size | 400 to 800 micron | 400 to 800 micron |

Cumulative Release Profile of Caffeine (%) over 12 hours

| Time (hr.) | Formula 12 | Formula 13 |
|---|---|---|
| 1 | 23 | 30 |
| 2 | 32 | 45 |
| 4 | 67 | 61 |
| 6 | 74 | 71 |
| 8 | 81 | 85 |
| 10 | 89 | 89 |
| 12 | 97 | 92 |

Process of Formula 1, as described in Example 1 was followed to prepare various caffeine formulations, covered in Table 4. The molten form was collected and processed to get granules in the range of 400 to 800 micron. The release profile was checked using dissolution apparatus, which is also included in Table 4.

The dissolution profile of caffeine formulation indicates prolonged release pattern, wherein about 25-30% of caffeine is released within first hour and about 60 to 75% of caffeine is released in 4-6 hours, followed by about 80-100% release in 8-12 hours. The release pattern is achieved through use of continuous phase of non-polymeric release controlling carriers.

1. A prolonged release caffeine formulation, comprising 20 to 70% by weight of caffeine, embedded in continuous phase of about 20 to 80% by weight of non-polymeric release controlling carrier, and optionally one or more excipients acceptable in nutraceutical and food industry.

2. Prolonged release caffeine formulation of claim 1, wherein the continuous phase is comprised of about 0 to 60% by weight of one or more excipients.

3. Prolonged release caffeine formulation of claim 2, wherein the continuous phase is comprised of about 0 to 50% by weight of one or more excipients.

4. Prolonged release caffeine formulation of claim 1, wherein the non-polymeric release controlling carrier is selected from lipids, fats, fatty acids, fatty alcohols, waxes, oils, their derivatives or the combination thereof.

5. Prolonged release caffeine formulation of claim 4, wherein the non-polymeric release controlling carrier is selected from carnauba wax, beeswax, stearic acid, cetyl alcohol, pegylated fatty acids, glycerol fatty acid esters, monoglycerides, diglycerides, triglycerides, glyceryl behenate, glyceryl monostearate, glyceryl palmitostearate, pegylated vegetable oils, partially hydrogenated oils of soy, cottonseed, palm, sunflower, castor oil, sorbitan esters, polyoxyethylene sorbitan esters, propylene glycol mono- or diesters, phospholipids, glycolipids, glycerolipid, sphingolipid, phosphatides, phosphatic acid, phosphatidylethanolamine, phosphatidylcholine, phosphatidylserine, phosphatidylinositol, glycerophospholipids, cerebrosides, gangliosides, cephalins, sulfatides, sugar esters, sugar ethers, sucrose esters, sterols, polyglycerol esters, ceramide, sterol, fat-soluble vitamins, prenol, saccharolipid, polyketides, their derivatives or the combination thereof.

6. Prolonged release caffeine formulation of claim 1, wherein one or more excipients may be selected from fillers, diluents, disintegrants, lubricants, binders, glidants, anti-caking agents, stabilizers, surfactants, channelizing agents, vehicles, buffers, stabilizers, preservatives, acidifiers, alkalizers, complexing agents, antioxidants, viscosity enhancers, plasticizers, coating materials, sweeteners, colors, flavors or the combination thereof.

7. Prolonged release formulation of claim 1, wherein about 25 to 60% of caffeine is released in first 1 hour, followed by about 50-90% of caffeine release in 4-8 hours and about 70 to 100% of release in 8-12 hours.

8. Process for preparation of prolonged release caffeine formulation, which is comprised of,
mixing caffeine with continuous phase of at least one non-polymeric release controlling carrier and optionally one or more excipients to get a blend,
subjecting the blend to suitable process of granulation by varying the temperature in the range of 50 to 100° C. to get molten form,
processing the molten form to get single-phase granules of desired size, and
formulating the granules in suitable dosage form.

9. Process for preparation of claim 8, wherein the granules of caffeine may vary in size ranging from 100 microns to 1500 microns.

10. Process for preparation of claim 8, wherein caffeine granules can be formulated in the form of sachets, mixed in beverages, filled in the capsules, compressed in the tablets, caplets, pills, beads, beadlets, pellets, formulated as chewing gum, gummies and the like.
